(19) United States
(12) Patent Application Publication
(10) Pub. No.: US 2006/0212012 A1
(43) Pub. Date: Sep. 21, 2006

(54) Absorbent core for a disposable obsorbent article

(76) Inventors: Alberto Corona Carlos, Puebla (MX); Carlos Canales Espinosa de les Monteros, Puebla (MX); Lucia Sanchez Fernandez, Puebla (MX); Fernando Rondero Pacheco, Puebla (MX)

Correspondence Address: Nelson Mullins Riley & Scarborough, LLP;17th Floor, 1320 Main Street, Columbia, SC, 29201, US

(21) Appl. No.: 11/344,563

(22) Filed: Jan. 31, 2006

(30) Foreign Application Priority Data
Feb. 2, 2005 (MX) PA/A/2005/001317

(51) Int. Cl. A61F 13/15

(52) U.S. Cl. 604/382; 604/370

(57) ABSTRACT

An absorbent core to be used in a disposable absorbent article is disclosed, the absorbent core being wrapped in a film of a non-woven fabric of any composition, with a basis weight of between about 10 and 15 g/m² and a pore size of between about 45 and 150 microns, such that there is an improvement in the characteristics of liquid return to the surface, integrity, softness, flexibility and lateral leakages thereof. Also disclosed is a disposable absorbent article containing same.

### BACKGROUND OF THE INVENTION

Generally, disposable absorbent articles, such as disposable diapers for babies, disposable diapers for incontinent persons, training pants or sanitary napkins, are basically comprised of a topsheet which permits passage of liquids, a liquid-impervious backsheet, and an absorbent core disposed between both of them; the absorbent core is intended for absorbing and containing the body exudates and is comprised, preferably, by fibers of an absorbent material and particles of a super-absorbent material.

The first disposable diapers that were offered in the market did not have the presently-known anatomic shape, since both the diaper and the absorbent core were rectangular in shape. Over time, the use of a wrapping paper to wrap the absorbent core in order to assist in the distribution of liquids and favor the integrity of the core became common. Also, the wrapping paper helped the handling of the core during the manufacturing process. When anatomic cores began to be used and upon improvement of the methods of shaping same, the use of wrapping paper was no longer necessary, and the latter was changed by one or more transfer layers disposed on one or both faces of the absorbent core, which, besides assisting the distribution of liquids, cooperate to prevent the return of same to the surface.

Presently, with the use of different densities and the combination of fibers and super-absorbent materials within the core, it has been possible to use again straight-shaped cores with an anatomic chassis; these cores are more compact and smaller, and also more comfortable to the user. With the use of straight cores it is possible again to use a wrapping layer with the absorbent core to help give body to same and to properly distribute liquids, the wrapping layer being cheaper than one or more transfer layers. On the other hand, it has been observed in practice that it is possible to use, instead of paper, a non-woven fabric to wrap the absorbent core which, depending on its characteristics, may have a cost equivalent to that of paper and also improve the liquid return characteristics, integrity, smoothness and containment of liquids, since said fabric may be hydrophobic in certain zones, such as those that will remain in the lateral edges of the core, as well as hydrophilic in the part or parts of same disposed over the core.

The present invention proposes the use of a non-woven fabric, with a basis weight of less than about 15 g/m², and a pore size of less than about 150 microns, to wrap the absorbent core of a disposable absorbent article and preferably treated such that the part or parts that will remain disposed on the core are hydrophilic, which the parts of same that will remain disposed to cover the lateral edges of same are hydrophobic, such that the fabric is as inexpensive as paper and provides the article with improved functionality characteristics.

There are some patents that disclose the use of a non-woven fabric with certain specific characteristics to wrap an absorbent core in articles of this type. As an example, U.S. Pat. No. 5,458,592 to Frank P. Abuto discloses an absorbent article whose core contains particles of a super-absorbent material and a non-woven fabric that wraps same, the purpose of which being to prevent leakage of super-absorbent material, to which end the non-woven fabric comprises a plurality of pores having a pore size lower than about 30 microns in order to make sure that the particles of super-absorbent material do not pass through same. On the other hand, published U.S. Patent Application 2003/0208176 A1, assigned to Tyco Healthcare, discloses a disposable absorbent article with a liquid-acquisition system comprising a non-woven fabric with a pair of marginal edge portions which are superposed on over the other, forming a double-layer fluid-acquisition system; the portions of the fabric that are superposed being treated to make them hydrophilic and the remainder of the fabric being hydrophobic.

In the present invention, with the use of a film of non-woven fabric with an average basis weight of less than about 15 g/m² and a pore size lower than about 150 microns as a wrapping layer for an absorbent core for a disposable absorbent article, characteristics such as liquid return to the surface of the core, integrity, smoothness, flexibility and leakage are substantially improved.

### OBJECTS OF THE INVENTION

On object of the present invention is to provide a soft and flexible absorbent core, to be used in a disposable absorbent article by means of including a film of a non-woven article as a wrapping layer for said core.

Another object of the invention consists of having the film of wrapping non-woven fabric help improve characteristics such as liquid return to the surface and integrity of the absorbent core.

An additional object of the invention resides in the film of wrapping non-woven fabric having hydrophobic characteristics in those parts thereof that will wrap the longitudinal edges of the core in order to prevent leakages.

Another object of the invention resides in the fact that the film of wrapping non-woven fabric helps to retain the super-absorbent material within the absorbent core.

A further object of the invention is that of obtaining a low-cost, soft, flexible disposable absorbent article, with low liquid return to the surface, good integrity and which retards leakages.

### BRIEF DESCRIPTION OF THE DRAWINGS

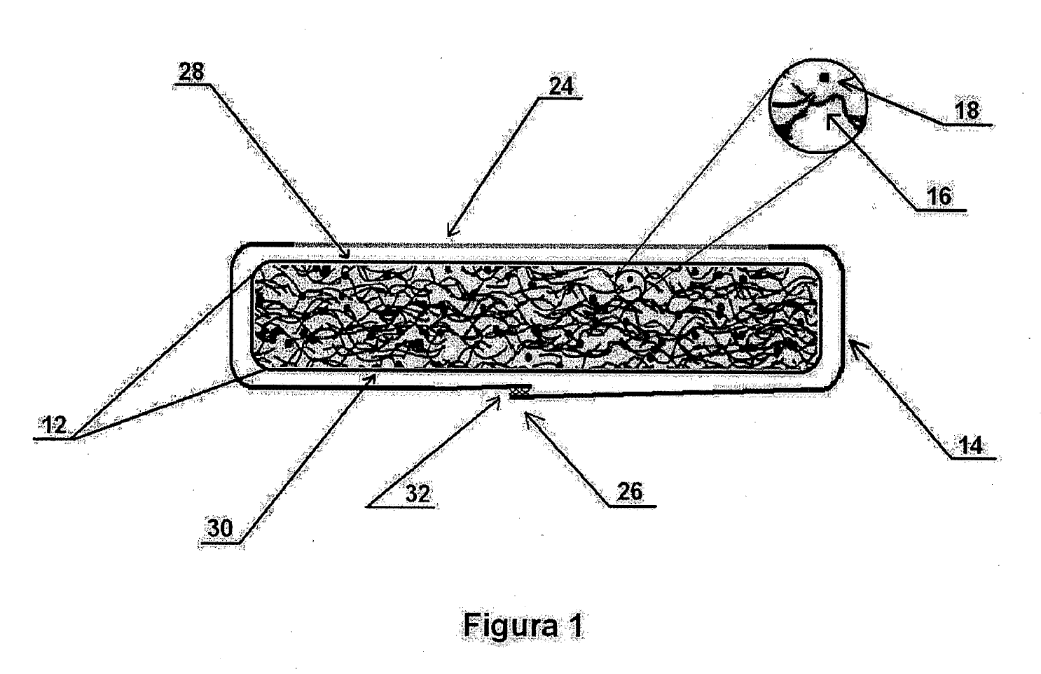

The FIG. 1 shows a cross section of the absorbent core of the present invention.

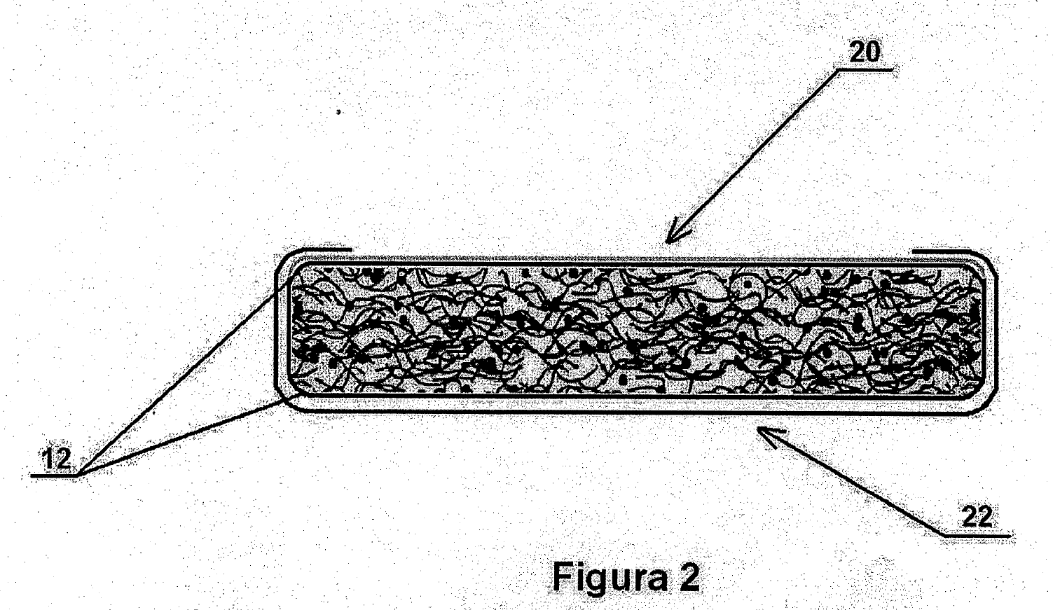

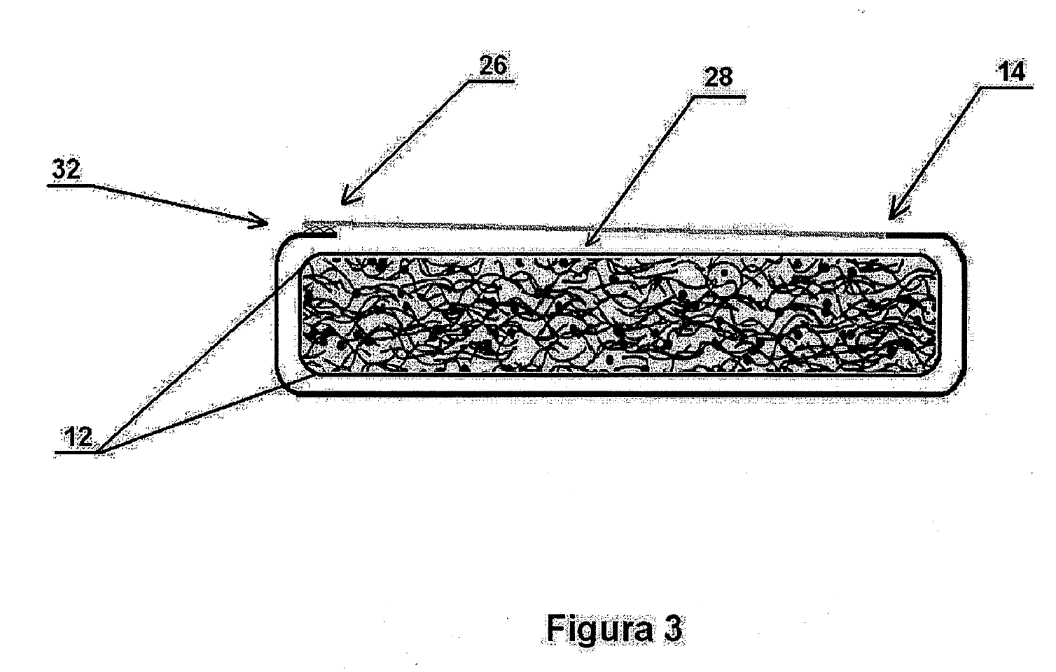

The FIGS. 2 and 3 show alternative embodiments of the absorbent core of the present invention.

### DETAILED DESCRIPTION OF THE INVENTION

A disposable absorbent article is an article that is used in proximity to the body to absorb and contain the body exudates and which is discarded after use. Some examples of this type of articles are disposable diapers, sanitary napkins and products for incontinent persons. The absorbent core of a disposable absorbent article is the part thereof intended for absorbing and containing the liquid and semi-liquid exudates which reach same; the core should also have the capability of distributing the liquids and retaining same, preventing as much as possible the return of the liquids to the surface of the article. On the other hand, the core must be soft and flexible, so that it conforms to the body of the user, without being uncomfortable to the user. The present invention relates to an absorbent core to be used in a disposable absorbent article, such as a baby diaper, an adult diaper, training pants, a sanitary napkin or similar articles, formed of an absorbent body which is wrapped in a film of a non-woven fabric that helps the absorbent body not to disintegrate or form clusters; the film also cooperates to prevent the return of liquids to the surface of the core, providing greater softness and flexibility, so that it conforms to and fits the body of the user. Preferably, the film of a non-woven fabric is treated such that at least the zones of same that cover the lateral parts of the absorbent body are hydrophobic, thus retarding the passage of liquids towards the lateral edges of the absorbent article, and the zone that remains on the absorbent body is hydrophilic; the film also helps prevent leakages of super-absorbent material from the core.

FIG. 1 shows a cross-section of a preferred embodiment of the absorbent core of the invention, which is formed by an absorbent body (12) and a film of non-woven fabric (14) which wraps this absorbent body. The absorbent body (12) has a top part (28) and a bottom part (30) and, preferably, is of a rectangular shape, containing fibers of absorbent material (16) and particles of a super-absorbent material (18). However, any type of absorbent body can be used, e.g., with different layers, with different densities within same and any shape and composition, provided only that it meets the purpose of absorbent cores. The wrapping non-woven fabric (14) overlaps and joins to itself in the bottom part of the absorbent body by means of a joint (32).

The non-woven fabric (14) which wraps the absorbent body (12) has a basis weight of between about 10 and 15 g/m², a pore size of between about 45 and 150 microns, and preferably is treated such that the zone (24) thereof that will be disposed on the top part (28) of the absorbent body (12) is hydrophilic, while the remainder of the fabric is hydrophobic. A non-woven fabric with these characteristics makes sure that the super-absorbent material has no possibilities of passing through same, since the size of the commonly-used super-absorbent material for this type of products varies between about 150 and 850 microns, there being a maximum of about 3% of the particles with a pore size of between about 45 and 150 microns.

With a non-woven fabric with a basis weight of between about 10 and 15 g/m², and a suitable forming process, it is possible to achieve a suitable distribution of fibers such that the return of liquids to the surface is minimum; on the other hand, water column values higher than about 100 mm in the hydrophobic zone can be achieved, which are sufficient to substantially retard the passage of liquids through same, all the foregoing with a cost similar to that of paper.

The wrapping non-woven fabric (14) overlaps by the bottom part of the core such that the absorbent body (12) is not exposed, such that the risk of exit of super-absorbent material from same is prevented, while providing the desired softness to the final product. The overlap (26) is the minimum possible overlap and is attached by means of the joint (32) by any known means, preferably by application of a hot-melt adhesive or any other suitable type of adhesive, as is know by those skilled in the art.

The non-woven fabric (14) can be comprised of natural fibers, synthetic fibers or one or more combinations of both; it can contain rayon, polyethylene, polyester or polypropylene, blends of these materials or it can be comprised only by one of these materials. It can be made by any method know in the art and be comprised of one or several layers joined to each other. Preferably, a 100% polypropylene SSS type of non-woven fabric is utilized; SSS means that the fabric is formed of three layers joined to each other, each of these formed by means of the method known in the art as “spunbond”. A film of this type assures a uniform distribution of the fibers within the fabric, such that the gram value can be reduced while keeping longitudinal and traverse strength, and also achieving a pore size in the non-woven fabric of between about 45 and 150 microns, such that the fabric can be used in the process of manufacture of a disposable absorbent article with no problem and while keeping the inexpensive the cost of the product but improving the functionality of the product and comfort for the user.

One of the functionality characteristics that are improved with the use of a wrapping non-woven fabric (14) of the present invention is the return of liquids to the surface; the Table below shows the results of laboratory tests of liquid return to the surface carried out with a paper-wrapped core (control), an absorbent core wrapped with 100% polypropylene non-woven fabric SSS with 10 g/m² basis weight (sample A) and an absorbent core wrapped with 100% polypropylene non-woven fabric SSS with 12 g/m² basis weight (sample B)

| | First Return | Second Return | Third Return |
|---|---|---|---|
| Control | 0.56 g | 6.41 g | 10.77 g |
| Sample A | 0.10 g | 0.30 g | 5.44 g |
| Sample B | 0.10 g | 0.37 g | 5.63 g |

For the tests, an absorbent core with an average weight of 29 g, containing about 60% by weight of absorbent fibers and about 40% by weight of super-absorbent particles was used.

As can be appreciated from the Table, the return of liquids, when a non-woven fabric is used as a wrapping layer, diminishes in more than about 80% for the first discharge, about 95% for the second discharge, and about 50% for the third discharge, values that indicate that this property is substantially improved with the use of a wrapping non-woven fabric having the described characteristics. On the other hand, there are no significant differences between the use of a non-woven fabric with a basis weight of about 10 g/m² and the use of a non-woven fabric with a basis weight of about 12 g/m².

In tests relating to use carried out in 50 babies and comparing diapers containing an absorbent core with a paper wrapping layer (control) and diapers containing an absorbent core with a wrapping film of 100% polypropylene non-woven fabric SSS with a basis weight of about 10 g/m² (inventive sample), the results show a substantial preference to the diapers of the inventive sample; in the specific test on the softness of the product, about 62% of the responses indicate that the diaper of the inventive sample is softer, both prior and after the use of the diapers. On the other hand, in the specific test on the integrity of the absorbent core, only about 3% of the responses indicate that the diaper of the inventive sample deformed, the deformation consisting of the fact that the core became narrower in the crotch section of the article, but in no case the core was split not it was separated, while in the control sample about 8.5% of the responses indicate that the panel was deformed and in about 5% of the cases the product was totally split. Thus, it is shown that an absorbent core with a wrapped non-woven fabric having the described characteristics has a better integrity and is softer than a paper-wrapped absorbent core.

Another of the functionality characteristics measured during the use test panel are those of leakages; the panel results show that in the diaper of the inventive sample about 5% of the cases showed leakage and only about 25% of these were through the longitudinal edges of the core. In the control sample, the leakages were present in about 13% of the cases, the leakages were by the longitudinal edges of the absorbent core in about 65% of the cases. It can thus be concluded that the absorbent core of the present invention helps diminish leakages in the longitudinal edges of the disposable absorbent article.

From the foregoing, it follows that with the use of a film of a non-woven fabric with the described characteristics to wrap an absorbent core, demonstrable functional advantages are achieved, such as a reduction in the liquid return to the surface, which is reflected in a higher dry feel in the skin of the user, a reduction in the deformation of the absorbent core during use, and a higher softness, which implies a greater flexibility and a better fit, both properties influencing for an increased comfort for the user, for reducing the presence of leakages through the longitudinal edge of the core, as well as preventing leakage of super-absorbent material. As a result, there is obtained a disposable absorbent article, such as a disposable diaper for babies, a disposable diaper for incontinent adults, a sanitary napkin, a panty-liner, training pants, and the like, with very good functionality and softness, at a low cost.

FIG. 2 shows an alternative embodiment of the present invention, in which, instead of using a wrapping non-woven fabric, an upper non-woven fabric (20) and a lower non-woven fabric (22) are used, such that the upper non-woven fabric (20) is hydrophilic and the lower non-woven fabric is hydrophobic, and having suitable dimensions to cover the longitudinal edges of the absorbent core (12), such as shown in FIG. 2.

FIG. 3 shows another alternative embodiment of the present invention, in which the overlap (26) of the wrapping non-woven fabric (14) of the present invention is carried out on the upper part (28) of the absorbent core (12), such that it is disposed adjacent the longitudinal edges of the absorbent body; the wrapping non-woven fabric (14) which is disposed on the upper part of the absorbent core is hydrophilic and the remainder of same is hydrophobic. In this manner, the overlap (26) preferably is made on the hydrophobic part of the fabric. It is not appropriate that the overlap (26) remain on the central upper part of the absorbent body (12), in the liquid-discharge zone, since when joining same by means of an adhesive, an hydrophobic zone is created in the joint; due to this reason, the overlap (26) of the fabric and the joint (32), if it remains on the upper part of the absorbent body (12), such as in the alternative shown in FIG. 3, it should be conveniently made adjacent one of the longitudinal edges thereof.

While the invention has been described as a function of its preferred embodiments, it is apparent that several changes and modifications thereto can be made, such changes and modifications falling within the spirit and scope of the invention, as defined in the attached claims.

1. An absorbent core to be used in a disposable absorbent article, formed of an absorbent body having two longitudinal edges and two transverse edges, an upper part and a lower part, and a film of a non-woven fabric which wraps same, characterized in that the wrapping non-woven fabric has a basis weight between about 10 and 15 g/m² and a pore size between about 45 and 150 microns.

2. An absorbent core to be used in a disposable absorbent article as recited in claim 1, characterized in that the wrapping film of a non-woven fabric overlaps on the lower part of the absorbent body.

3. An absorbent core to be used in a disposable absorbent article as recited in claim 1, characterized in that the wrapping film of a non-woven fabric overlaps on the upper part of the absorbent body, adjacent one of the longitudinal edges thereof.

4. An absorbent core to be used in a disposable absorbent article as recited in the preceding claims, characterized in that it is treated such that the part or parts that are disposed on the absorbent body are hydrophilic and the remainder is hydrophobic, the hydrophobic zone having a minimum water column value of about 100 mm.

5. An absorbent core to be used in a disposable absorbent article as recited in the preceding claims, characterized in that the wrapping film of a non-woven fabric is 100% polypropylene.

6. An absorbent core to be used in a disposable absorbent article as recited in the preceding claims, characterized in that the wrapping film of a non-woven fabric is formed by the attachment of three films of spunbond type material.

7. An absorbent core to be used in a disposable absorbent article, formed of an absorbent body having two longitudinal edges and two transverse edges, an upper part and a lower part and two films of non-woven fabric, characterized in that the first film of non-woven fabric covers the uppers part of the absorbent body and the second film of non-woven fabric covers the lower part of at least the longitudinal edges thereof, such that both films of non-woven fabric have a weight basis of between about 10 and 15 g/m² and a size pore of between about 45 and 150 microns.

8. An absorbent core to be used in a disposable absorbent article as recited in claim 7, characterized in that the second film of non-woven fabric is hydrophobic and has a minimum water column value of about 100 mm.

9. A disposable absorbent article, comprising topsheet, a backsheet, and an absorbent core disposed between both the topsheet and the backsheet, characterized in that the absorbent core is formed of an absorbent body wrapped in film of a non-woven fabric of any composition, having a basis weight of between about 10 and 15 g/m² and a pore size of between about 45 and 150 microns.

10. A disposable absorbent article as recited in claim 9, characterized in that the film of a non-woven fabric is treated such that the part or parts thereof that are disposed on the absorbent core and hydrophilic and the remainder thereof is hydrophobic, the hydrophobic zone 2 having a minimum water column value of about 100 mm.

11. A disposable absorbent article, comprising a topsheet, a backsheet, and an absorbent core disposed between both the topsheet and the backsheet, characterized in that the absorbent core is formed of an absorbent body and two films of a non-woven fabric, a first film covering the upper part of the absorbent body and a second film covering the lower or bottom part and at least the longitudinal edges thereof, such that both films have a basis weight of between about 10 and 15 g/m² and a pore size of between about 45 and 150 microns.

12. A disposable absorbent article as recited in claim 11, characterized in that the first film is hydrophilic and the second film is hydrophobic, the latter having a minimum water column value of about 100 mm.

13. A disposable absorbent article as recited in claims 9 through 12, characterized in that the article is for feminine hygiene.

14. A disposable absorbent article as recited in claims 9 through 12, characterized in that the article is a training pant.

15. A disposable absorbent article as recited in claims 9 through 12, characterized in that the article is a disposable diaper.

16. A disposable absorbent article as recited in claims 9 through 12, characterized in that the article is an article for incontinent persons.
